(19) United States
(12) Patent Application Publication
(10) Pub. No.: US 2013/0131375 A1
(43) Pub. Date: May 23, 2013

(54) PROCESS FOR PREPARING DIFLUOROACETIC ACID, SALTS THEREOF OR ESTERS THEREOF

(71) Applicant: Olivier Buisine, Saint Genis-Laval (FR)

(72) Inventor: Olivier Buisine, Saint Genis-Laval (FR)

(73) Assignee: RHODIA OPERATIONS, Aubervilliers (FR)

(21) Appl. No.: 13/814,471

(22) PCT Filed: Jul. 26, 2011
(86) PCT No.: PCT/EP11/62779

§ 371 Date: Feb. 5, 2013

(30) Foreign Application Priority Data
Aug. 5, 2010 (FR) 10/03283

(51) Int. Cl. C07C 67/287; C07C 51/363

(52) U.S. Cl. CPC C07C 67/287; C07C 51/363
USPC 560/226; 562/603

(57) ABSTRACT

A process for preparing difluoroacetic acid, salts thereof or esters thereof is described. The process can further include preparation of difluoroacetic acid, salts thereof or esters thereof, wherein the reaction occurs in the presence of water of a salt providing a fluoride anion and of monohalogenated or dihalogenated acetic acid, in acid, salified or esterified form, at least one halogen atom being other than the fluorine atom.

The subject of the present invention is a process for preparing difluoroacetic acid, salts thereof or esters thereof.

More specifically, the invention relates to a process for preparing said compounds according to a halogen atom exchange reaction.

It is known from JP-A 06-228043 how to prepare difluoroacetic acid according to a reaction between an N,N-dichloroacetamide and potassium fluoride, in glycol at 150° C.

The drawback of the process described is that it involves a substrate of amide type.

In EP 0 694 523, the preparation of the fluoride of difluoroacetic acid or esters thereof, by reaction of a 1-alkoxy-1,1,2,2-tetrafluoroethane, in the gas phase, in the presence of a catalyst of metal oxide type, is described.

This process has the disadvantage of requiring a gaseous substrate which is explosive in air.

In order to overcome the aforementioned drawbacks, the invention proposes a completely different process.

Thus, a process has now been found, and it is this which constitutes the subject of the present invention, for preparing difluoroacetic acid, salts thereof or esters thereof, characterized in that it comprises the reaction in the presence of water of a salt providing a fluoride anion and of monohalogenated or dihalogenated acetic acid, in acid, salified or esterified form; at least one halogen atom being other than a fluorine atom.

In accordance with the process of the invention, monohalogenated or dihalogenated acetic acid or one of the salts or esters thereof comprising at least one halogen atom other than a fluorine atom is subjected to a halogen/fluorine exchange reaction.

In the present text, the term “halogen” is understood to mean chlorine or bromine.

For the simplification of the account of the invention, said compound will be denoted in a simplified manner by “halogenated substrate”.

The starting substrate may be in acid form. It is then understood to be monohalogenated or dihalogenated acetic acid with at least one halogen atom other than a fluorine atom.

The starting substrate may be in salified form. In this case, the aforementioned acid for which the hydrogen atom is replaced by a metal cation is denoted.

The starting substrate may be in esterified form. In this case, the aforementioned acid for which the hydrogen atom is replaced by a hydrocarbon-based group, preferably an alkyl or cycloalkyl group, is denoted.

A halogenated substrate is involved in the process of the invention, which halogenated substrate may preferably be represented by the following formula:

HCX₁X₂—COOR₁ (I)

in said formula:
X₁ and X₂, which are identical or different, represent a chlorine, bromine or fluorine atom with the condition that at least one of the X₁ or X₂ atoms is a chlorine or bromine atom;
R₁ represents:
   a hydrogen atom;
   a substituted or unsubstituted hydrocarbon-based group, which may be an alkyl or cycloalkyl group;
   a metal cation.

Within the context of the invention, the term “alkyl” is understood to mean a linear or branched hydrocarbon-based chain having from 1 to 15 carbon atoms and preferably from 1 or 2 to 10 carbon atoms.

Examples of preferred alkyl groups are in particular methyl, ethyl, propyl, isopropyl, butyl, isobutyl and t-butyl.

The term “cycloalkyl” is understood to mean a monocyclic cyclic hydrocarbon-based group comprising from 3 to 8 carbon atoms, preferably a cyclopentyl or cyclohexyl group.

It should be noted that, in these groups, one or more hydrogen atoms may be replaced with a substituent (for example halogen), as long as it does not interfere with obtaining the desired product.

In particular, the hydrocarbon-based chain may preferably bear one or more fluorine atoms.

Thus, R₁ may represent a fluorinated or perfluorinated alkyl group comprising from 1 to 10 carbon atoms and from 1 to 21 fluorine atoms, preferably from 3 to 21 fluorine atoms.

In the formula (I), R₁ represents a hydrogen atom and preferably an alkyl group having from 1 to 4 carbon atoms.

R₁ preferably represents a methyl or ethyl group.

R₁ also represents a metal cation, preferably a cation of a monovalent or divalent metal.

Mention may more particularly be made, preferably, of an alkali or alkaline-earth metal cation.

As more specific examples of salts, mention may be made of alkali metal cations, preferably lithium, sodium, potassium or caesium; and alkaline-earth metal cations, preferably magnesium, calcium or barium.

In the aforementioned list, the preferred metal cations are sodium or potassium cations.

The halogenated substrates preferably used in the process of the invention are monochloroacetic acid, dichloroacetic acid, chlorofluoroacetic acid or the methyl or ethyl esters thereof.

As regards the salt providing the fluoride anion, use may be made of a metal fluoride and more particularly of fluorides of metals from groups (IA), (IIA) or (IIB) of the Periodic Table of the Elements.

In the present text, reference is made hereinbelow to the Periodic Table of the Elements published in the Bulletin de la Société Chimique de France [Bulletin of the French Chemical Society], No. 1 (1966).

As examples of cations that are particularly suitable for the process of the invention, mention may more particularly be made, among those from group (IA), of lithium, sodium, potassium and caesium; from group (IIA), of magnesium and calcium; and from group (IIB), preferably of zinc.

Among the aforementioned salts, potassium fluoride is preferably chosen.

Potassium bifluoride KHF₂ may also be used.

The invention does not exclude the use of double salts such as double fluorides of aluminum and of sodium or potassium; and sodium or potassium fluorosilicates.

As examples of other salts providing fluoride ions, mention may also be made of onium fluorides and more particularly the fluorides of ammonium and of phosphonium for which the cation corresponds, in particular, to the following formula:

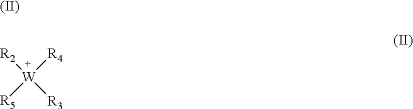

in said formula:
W represents N or P;
R₂, R₃, R₄ and R₅, which are identical or different, represent:
   a linear or branched alkyl group having from 1 to 16 carbon atoms and that is optionally substituted by one or more phenyl, hydroxyl, halogen, nitro, alkoxy or alkoxycarbonyl groups or atoms, the alkoxy groups having from 1 to 4 carbon atoms;
   a linear or branched alkenyl group having from 2 to 12 carbon atoms;
   an aryl group having from 6 to 10 carbon atoms, optionally substituted by one or more alkyl having from 1 to 4 carbon atoms, alkoxy or alkoxycarbonyl, the alkoxy group having from 1 to 4 carbon atoms, or halogen groups or atoms.

The fluorides preferably used have a cation that corresponds to the formula (II) in which W is a nitrogen or phosphorus atom and R₂, R₃, R₄ and R₅, which are identical or different, represent a linear or branched alkyl group having from 1 to 4 carbon atoms and a benzyl group.

As more specific examples, mention may be made of tetrabutylammonium, methyltri(n-butyl)ammonium, N-methyl-N,N,N-trioctylammonium, trimethylphenylphosphonium, tetrabutylphosphonium, methyltri(n-butyl)phosphonium, methyltri(isobutyl)phosphonium and diisobutyl-(n-octyl)methylphosphonium fluorides.

Preferably, tetrabutylammonium fluoride or tetrabutylphosphonium fluoride is chosen.

As other salts that provide a fluoride, mention may be made of those for which the cation corresponds to one of the following formulae:

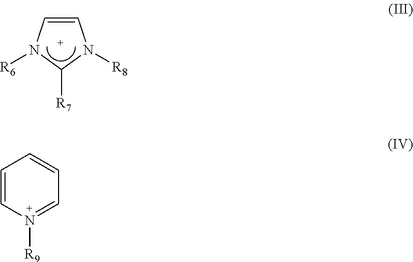

in said formulae:
the R₆ group represents an alkyl group having from 1 to 20 carbon atoms;
the R₇ group represents a hydrogen atom or an alkyl group having from 1 to 4 carbon atoms;
the R₈ group represents an alkyl group having from 1 to 4 carbon atoms; and
the R₉ group represents an alkyl group having from 1 to 6 carbon atoms.

Among the cations corresponding to the formulae (III) and (IV), mention may be made of the cations:
1-alkyl-2,3-dimethylimidazolium;
1-alkyl-3-methylimidazolium; and
1-alkylpyridinium.

As more specific examples of onium salts, mention may be made of 1-alkyl-2,3-dimethylimidazolium fluorides such as 1-ethyl-2,3-dimethylimidazolium fluoride, 1-butyl-2,3-dimethylimidazolium fluoride or 1-hexyl-2,3-dimethylimidazolium fluoride; 1-butyl-2,3-dimethylimidazolium tetrafluoroborate or 1-hexyl-2,3-dimethylimidazolium tetrafluoroborate; 1-alkyl-3-methylimidazolium fluorides such as 1-ethyl-3-methylimidazolium fluoride, 1-hexyl-3-methylimidazolium fluoride, 1-octyl-3-methylimidazolium fluoride, 1-decyl-3-methylimidazolium fluoride, 1-dodecyl-3-methylimidazolium fluoride, 1-tetradecyl-3-methylimidazolium fluoride, 1-hexadecyl-3-methylimidazolium fluoride or 1-octadecyl-3-methylimidazolium fluoride; 1-butyl-3-methylimidazolium hexafluorophosphate, 1-hexyl-3-methylimidazolium hexafluorophosphate or 1-octyl-3-methylimidazolium hexafluorophosphate; 1-butyl-3-methylimidazolium tetrafluoroborate or 1-hexyl-3-methylimidazolium tetrafluoroborate; 1-alkylpyridinium salts such as 1-ethylpyridinium fluoride, 1-butylpyridinium fluoride or 1-hexylpyridinium fluoride; 1-butylpyridinium hexafluorophosphate or 1-hexylpyridinium hexafluorophosphate; or 1-butylpyridinium tetrafluoroborate or 1-hexylpyridinium tetrafluoroborate.

1-Butyl-3-methylimidazolium hexafluorophosphate or 1-butyl-3-methylimidazolium tetrafluoroborate is preferably chosen.

The invention does not exclude the use of chloride or bromide halogenated precursors, it being possible for the corresponding fluorides to be formed in situ, by reaction with a metal fluoride as defined previously, preferably potassium fluoride.

Use may also be made, in the process of the invention, of a mixture of the various salts that provide a fluoride anion.

According to one variant of the process of the invention, it is possible to use a fluoride provided by a salt, for example potassium fluoride, and to add an onium salt as defined previously.

In this case, the amount of onium fluoride (or of the precursor thereof) may represent from 1 to 10 mol % expressed relative to the substrate of formula (I).

According to the process of the invention, the reaction between the halogenated substrate of formula (I) and the salt providing the fluoride anion is carried out in the presence of water.

The ratio between the number of moles of salt expressed as fluoride anion and the number of moles of halogenated substrate of formula (I) may vary between 2 and 10, and preferably lies between 5 and 6.

The exchange reaction is carried out in the presence of water. The amount of water in the reaction mixture is such that it represents from 1 to 90% of the weight thereof. The expression “reaction mixture” is understood to mean the halogenated substrate, the salt providing the fluoride anion, the water and optionally an organic solvent.

Indeed, the reaction may be carried out in an aqueous medium or in an aqueous-organic medium. The organic solvent is advantageously a polar protic solvent.

As preferred examples of polar protic solvents, mention may be made of alcohols.

As examples of alcohols, mention may be made of aliphatic primary alcohols having from 1 to 5 carbon atoms.

Methanol and ethanol are the preferred solvents.

Use may also be made of a mixture of alcohols.

The amount of alcohol used is such that the water/alcohol mixture has the following composition:
from 1 to 99% by weight of water; and
from 99 to 1% by weight of alcohol.

The exchange reaction is generally carried out at a temperature between 80° C. and 120° C. when it is conducted at atmospheric pressure.

The temperature is preferably chosen between 95° C. and 105° C.

The reaction may be carried out at higher temperature, for example between 100° C. and 150° C., under autogenous pressure of the reactants.

The exchange reaction is generally preferably carried out under a controlled atmosphere of inert gases. An atmosphere of noble gases, preferably argon, may be established, but it is more economical to use nitrogen.

The process of the invention is simple to implement.

The reactants may be introduced in any order according to various variants, but certain ones are preferred.

One preferred embodiment consists in mixing the water, optionally the organic solvent, preferably alcohol, and the halogenated substrate and then in introducing the salt providing the fluoride anion in one go or gradually, in fractions or continuously.

According to one variant of the process of the invention, the pH is advantageously adjusted during the reaction to a value of less than 10, preferably of less than 9 and preferably selected between 5 and 9 and very preferably between 7 and 9.

The pH may be adjusted in particular using hydrofluoric acid or a basic aqueous solution, preferably a solution of sodium hydroxide or potassium hydroxide, the concentration of which advantageously varies between 40 and 70% by weight.

The reaction mixture is brought, with stirring, to the chosen reaction temperature within the range as defined previously.

The heating of the reaction mixture is maintained for a variable duration. By way of example, it is specified that the duration of the reaction carried out at 100° C. generally varies between 10 hours and 40 hours.

After keeping the reaction medium stirred, at the chosen temperature, at the end of the reaction difluoroacetic acid, salts thereof or esters thereof are obtained that correspond to the following formula:

H—CF₂—COOR₁ (V)

in said formula, R₁ has the meaning given previously.

The compound of formula (V) may be recovered from the reaction mixture in particular by the separation technique described in WO 2010/03986.

The process of the invention is advantageously carried out in apparatus capable of withstanding the corrosion of the reaction medium.

For this purpose, corrosion-resistant materials are chosen for the part in contact with the reaction medium, such as alloys based on molybdenum, chromium, cobalt, iron, copper, manganese, titanium, zirconium, aluminum, carbon and tungsten sold under the HASTELLOY® trade marks or alloys of nickel, chromium, iron, manganese to which copper and/or molybdenum are added sold under the name INCONEL® and more particularly the HASTELLOY C 276 or INCONEL 600, 625 or 718 alloys.

Stainless steels may also be chosen, such as austenitic steels [Robert H. Perry et al., Perry's Chemical Engineers' Handbook, Sixth Edition (1984), pages 23-44] and more particularly the 304, 304 L, 316 or 316 L stainless steels. A steel having a nickel content of at most 22% by weight, preferably of between 6 and 20% and more preferably of between 8 and 14% is used.

The 304 et 304 L steels have a nickel content that varies between 8 and 12% and the 316 and 316 L steels have a nickel content that varies between 10 and 14%.

More particularly, 316 L steels are chosen.

Use may also be made of equipment constituted of or coated with a polymeric compound resistant to the corrosion of the reaction medium. Mention may especially be made of materials such as PTFE (polytetrafluoroethylene or Teflon) or PFA (perfluoroalkyl resins) or high-density polyethylene. It will not be outside the scope of the invention to use an equivalent material.

As other materials capable of being suitable for being in contact with the reaction medium, mention may also be made of derivatives of graphite.

The process of the invention may be carried out continuously or in batch mode.

It is particularly advantageous since it is a simple process which does not require anhydrous conditions and does not necessitate the use of toxic solvents.

Exemplary embodiments of the invention are given hereinbelow. These examples are given by way of illustration and non-limitingly.

In the examples, the degree of conversion and the yield obtained are defined.

The degree of conversion (TT) corresponds to the ratio between the number of moles of dichloroacetic acid or ester thereof that are converted and the number of moles of dichloroacetic acid or ester thereof that are used.

The yield (RR) corresponds to the ratio between the number of moles of difluoroacetic acid or ester thereof that are formed and the number of moles of dichloroacetic acid or ester thereof that are used.

### EXAMPLES 1 to 4

Given below is the procedure which will be taken up in the various examples 1 to 4.

A solution of dichloroacetic acid or of the ethyl ester thereof in water or as a mixture with ethanol is charged to a glass reactor.

Solid potassium fluoride is added and the temperature of the medium is brought to 100° C. for a duration of 40 hours.

After returning to ambient temperature, the aqueous solution is assayed by ¹H NMR and ¹⁹F NMR.

All of the operating conditions and results obtained are listed in Table (I) below:

TABLE (I)

| | | | KF | Solvent | | |
|---|---|---|---|---|---|---|
| | HCl₂CCO₂R₁ | HCl₂CCO₂R₁ | Weight | Volume | | |
| c | R₁= | Weight (g) | (g) | (mL) | TT_{HCl2CCO2R1} % | RR_{HCF2CO2R1} % |
| 1 | H | 1 | 2.86 | H₂O (10 mL) | 74 | 24 |
| 2 | H | 1 | 2.81 | H₂O(5 mL), | 43 | 33 |
| | | | | EtOH(5 mL) | | |
| 3 | Et | 1.227 | 2.71 | H₂O(5 mL), | 36 | 21 |
| | | | | EtOH(5 mL) | | |
| 4 | Et | 1.271 | 2.88 | EtOH/H₂O* | 87 | 5 |
| | | | | (10 mL) | | |

*In Example 4, the water represents 1% of the weight of the EtOH/H₂O mixture.

### EXAMPLE 5

In this example, chlorofluoroacetic acid is used as halogenated substrate.

The chlorofluoroacetic acid (5 g) in solution in 50 g of water is brought into contact with potassium fluoride (15 g) and the mixture is brought to a temperature of 100° C. for a duration of 22 hours.

The aqueous solution is then analysed by ¹H NMR and ¹⁹F NMR.

The degree of conversion of the chlorofluoroacetic acid is 100%.

The yield of difluoroacetic acid is 41%.

### EXAMPLES 6 to 9

Given hereinbelow is the procedure which will be taken up in the following various examples 6 to 9.

A solution of dichloroacetic acid in water is charged to a glass reactor.

A solid fluorinating agent is added and the temperature of the medium is brought to 120° C. for 16 hours.

After returning to ambient temperature, the aqueous solution is assayed by ¹H NMR and ¹⁹F NMR.

All of the operating conditions and results obtained are listed in Table (II) below:

TABLE (II)

| | Fluorinating | HCl₂CCO₂H | Fluoride | Water | | |
|---|---|---|---|---|---|---|
| Example | agent | Weight (g) | equivalents | (mL) | TT_{HCl2CCO2H} % | RR_{HCF2CO2H} % |
| 6 | CaF₂ | 5 | 6 | 10.5 | 98 | 18 |
| 7 | NH₄F | 5 | 6 | 10.5 | 100 | 24 |
| 8 | NaF | 5 | 6 | 10.5 | 100 | 23 |
| 9 | KHF₂ | 5 | 6 | 10.5 | 100 | 21 |

### EXAMPLES 10 to 12

Given below is the procedure which will be taken up in the following various examples 10 to 12.

Added to a solution of KF in water brought to a temperature of 120° C. is dichloroacetic acid (2 g). The medium is left stirring at 120° C. for 1 hour.

After returning to ambient temperature, the aqueous solution is assayed by ¹H NMR and ¹⁹F NMR.

All of the operating conditions and results obtained are listed in Table (III) below:

TABLE (III)

| | Weight of | Weight of | TT_{HCl2CCO2H} | RR_{HCF2CO2H} |
|---|---|---|---|---|
| Example | KF (g) | water (mL) | % | % |
| 10 | 3.6 | 2.8 | 100 | 44 |
| 11 | 7.2 | 5.6 | 100 | 89 |
| 12 | 18 | 14 | 100 | 96 |

### EXAMPLE 13

Added to a solution of KF (18 g) in 14 mL of water brought to a temperature of 150° C. is dichloroacetic acid (2 g). The medium is left stirring at 150° C. for 6 minutes.

After returning to ambient temperature, the aqueous solution is assayed by ¹H NMR and ¹⁹F NMR.

The degree of conversion of the dichloroacetic acid is 100%.

The yield of difluoroacetic acid is 94%.

1. A process for preparing difluoroacetic acid, salts thereof or esters thereof, the process comprising conducting a reaction in the presence of water of a salt providing a fluoride anion and of monohalogenated or dihalogenated acetic acid, in acid, in salified or esterified form; at least one halogen atom being other than a fluorine atom.

2. The process as defined by claim 1, wherein the halogenated substrate corresponds to the following formula:
HCX₁X₂—COOR₁ (I)
in said formula:
X₁ and X₂, which are identical or different, represent a chlorine, bromine or fluorine atom with the condition that at least one of the X₁ or X₂ atoms is a chlorine or bromine atom; and
R₁ represents:
a hydrogen atom;
a substituted or unsubstituted hydrocarbon-based group, which is optionally an alkyl or cycloalkyl group; or
a metal cation.

3. The process as defined by claim 2, wherein the halogenated substrate corresponds to the formula (I) in which:
R₁ represents a hydrogen atom;
R₁ represents an alkyl group having from 1 to 4 carbon atoms; and
R₁ represents an alkali or alkaline-earth metal cation.

4. The process as defined by claim 1, wherein the halogenated substrate is monochloroacetic acid, dichloroacetic acid, chlorofluoroacetic acid or the methyl or ethyl esters thereof.

5. The process as defined by claim 1, wherein the salt providing the fluoride anion is one of the following salts or mixtures thereof:
a metal fluoride,
a double salt,
an onium fluoride (II); and
an onium fluoride (III) or (IV).

6. The process as defined by claim 5, wherein the salt providing the fluoride anion is potassium fluoride or potassium bifluoride KHF₂.

7. The process as defined by claim 5, wherein use is made of a fluoride provided by a salt, and an onium fluoride or one of the precursors thereof.

8. The process as defined by claim 1, wherein the ratio between the number of moles of salt expressed as fluoride anion and the number of moles of halogenated substrate of formula (I) varies from 2 to 10.

9. The process as defined by claim 1, wherein the exchange reaction is carried out in the presence of water: the amount of water in the reaction mixture being such that it represents from 1% to 90% of the weight thereof.

10. The process as defined by claim 1, wherein the reaction takes place in an aqueous-organic medium: the organic solvent being a polar protic solvent.

11. The process as defined by claim 10, wherein the organic solvent is an aliphatic primary alcohol having from 1 to 5 carbon atoms.

12. The process as defined by claim 10, wherein the amount of alcohol used is such that the water/alcohol mixture has the following composition:
from 1% to 99% by weight of water; and
from 99% to 1% by weight of alcohol.

13. The process as defined by claim 1, wherein the exchange reaction is carried out at atmospheric pressure at a temperature of from 80° C. to 120° C.

14. The process as defined by claim 1, wherein the exchange reaction is carried out from 100° C. to 150° C., under autogenous pressure of the reactants.

15. The process as defined by claim 1, wherein the process further comprises mixing the water, optionally the organic solvent, and the halogenated substrate and, then in introducing the salt providing the fluoride anion at once or gradually, in fractions or continuously.

16. The process as defined by claim 15, wherein the pH is adjusted during the reaction to a value of less than 10.

17. The process as defined by claim 1, wherein the reaction mixture is brought, with stirring, to the chosen reaction temperature.

18. The process as defined by claim 2, wherein at the end of the reaction difluoroacetic acid, salts thereof or esters thereof are obtained that correspond to the formula:
H—CF₂—COOR₁ (V).

19. The process as defined by claim 5, wherein the metal fluoride is a fluoride of a metal from Groups (IA), (IIA) or (IIB) of the Periodic Table of the Elements.

20. The process as defined by claim 5, wherein the double salt is a double fluoride of aluminum and of sodium or potassium, and a sodium or potassium fluorosilicate.

21. The process as defined by claim 5, wherein the onium fluoride (II) is a fluoride of ammonium and of phosphonium for which the cation corresponds to the following formula: 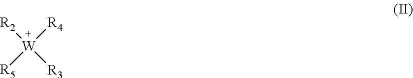
in said formula:
W represents N or P;
R₂, R₃, R₄ and R₅, which are identical or different, represent:
a linear or branched alkyl group having from 1 to 16 carbon atoms and that is optionally substituted by one or more phenyl, hydroxyl, halogen, nitro, alkoxy or alkoxycarbonyl groups or atoms, the alkoxy groups having from 1 to 4 carbon atoms;
a linear or branched alkenyl group having from 2 to 12 carbon atoms; and
an aryl group having from 6 to 10 carbon atoms, optionally substituted by one or more alkyl having from 1 to 4 carbon atoms, alkoxy or alkoxycarbonyl, the alkoxy group having from 1 to 4 carbon atoms, or halogen groups or atoms.

22. The process as defined by claim 5, wherein the onium fluoride (III) or (IV) is an imidazolinium or pyridinium fluoride, the cation of which corresponds to the formulae: 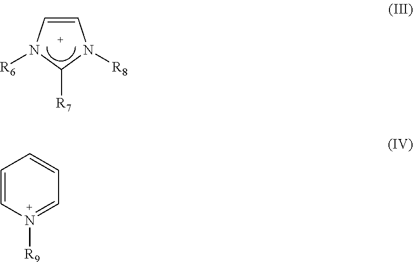
in said formulae:
the R₆ group represents an alkyl group having from 1 to 20 carbon atoms;
the R₇ group represents a hydrogen atom or an alkyl group having from 1 to 4 carbon atoms;
the R₈ group represents an alkyl group having from 1 to 4 carbon atoms; and
the R₉ group represents an alkyl group having from 1 to 6 carbon atoms.

23. The process as defined by claim 7, wherein the fluoride provided by the salt is potassium fluoride.

24. The process as defined by claim 8, wherein the ratio of moles varies from 5 to 6.

25. The process as defined by claim 11, wherein the aliphatic primary alcohol is methanol or ethanol.

26. The process as defined by claim 13, wherein the reaction temperature is from 95° C. to 105° C.

27. The process as defined by claim 15, wherein the organic solvent is alcohol.

28. The process as defined by claim 16, wherein pH is adjusted to a value of less than 9.

29. The process as defined by claim 16, wherein the pH is adjusted to a value of from 7 to 9.
